(19) United States
(12) Patent Application Publication
(10) Pub. No.: US 2018/0033142 A1
(43) Pub. Date: Feb. 1, 2018

(54) IMAGE-PROCESSING APPARATUS, BIOLOGICAL OBSERVATION APPARATUS, AND IMAGE-PROCESSING METHOD

(71) Applicant: OLYMPUS CORPORATION, Tokyo (JP)

(72) Inventor: Yasunori MORITA, Tokyo (JP)

(73) Assignee: OLYMPUS CORPORATION, Tokyo (JP)

(21) Appl. No.: 15/723,255

(22) Filed: Oct. 3, 2017

Related U.S. Application Data
(63) Continuation of application No. PCT/JP2015/060748, filed on Apr. 6, 2015.

(51) Int. Cl. G06T 7/00; A61B 1/06; A61B 5/00; A61B 1/04; A61B 1/00

(52) U.S. Cl. CPC G06T 7/0012; A61B 1/04; A61B 1/00045; A61B 5/4872; A61B 1/0638; A61B 5/7203; A61B 5/7221; A61B 5/7278; A61B 2576/00; G06T 2207/10068; G06T 2207/30101

(57) ABSTRACT

Provided is an image-processing portion including: a fat-region setting portion that detects fat-region information that indicates a fat region in which fat exists in a biological-tissue image; a blood-region-information detecting portion that detects blood-region information that indicates a blood region in which blood exists in the biological-tissue image; a confidence-calculating portion that calculates a confidence for the fat-region information on a basis of the fat-region-information detected by the fat-region setting portion and the blood-region-information detected by the blood-region-information detecting portion; and a display-form setting portion that manipulates the fat region indicated by the fat-region information for which the calculated confidence calculated by the fat-region confidence-calculating portion is lower than a reference confidence that serves as a reference so as to have a display form that can be distinguished from a peripheral region.

### CROSS-REFERENCE TO RELATED APPLICATIONS

This is a continuation of International Application PCT/JP2015/060748, with an international filing date of Apr. 6, 2015, which is hereby incorporated by reference herein in its entirety.

### TECHNICAL FIELD

The present invention relates to an image-processing apparatus, a biological observation apparatus, and an image-processing method.

### BACKGROUND ART

In the related art, there is a known narrow-band imaging (NBI) technique in which illumination light that has been converted to have a narrow-band wavelength which is more easily absorbed by hemoglobin contained in blood is radiated, whereby the capillaries in a mucosal surface or the like are displayed in an emphasized manner (for example, see Patent Literature 1).

This narrow-band imaging is expected to serve as an alternative observation method to dye spraying that is widely practiced in order to perform detailed diagnosis of an esophagus region or to observe the pit pattern (ductal structure) of the colon, and is expected to contribute to increasing the efficiency of examinations by decreasing the duration of examinations and unnecessary biopsies

### CITATION LIST

### Patent Literature

### {PTL 1} Japanese Unexamined Patent Application, Publication No. 2011-224038

### {PTL 2} PCT International Publication No. 2013/115323

### SUMMARY OF INVENTION

### Technical Problem

An object of the present invention is to provide an image-processing apparatus, a biological observation apparatus, and an image-processing method with which it is possible to reduce the risk of damaging the nerves by allowing a surgeon to ascertain a region in which fat detection is hindered due by the influence of blood and other disturbances, thus making it impossible to accurately detect fat.

### Solution to Problem

A first aspect of the present invention is an image-processing apparatus including: a fat-region-information detecting portion that detects fat-region information that indicates a fat region in which fat exists in a biological-tissue image; a blood-region-information detecting portion that detects blood-region information that indicates a blood region in which blood exists in the biological-tissue image, a confidence-calculating portion that calculates a confidence for the fat-region information on a basis of the fat-region-information detected by the fat-region-information detecting portion and the blood-region-information detected by the blood-region-information detecting portion; and a display-form manipulating portion that manipulates the fat region indicated by the fat-region information for which the calculated confidence calculated by the confidence-calculating portion is lower than a reference confidence that serves as a reference so as to have a display form that can be distinguished from a peripheral region.

In the above-described aspect, the calculated confidence may be increased with an increase in an SN ratio of the fat-region information and may be decreased with a decrease in the SN ratio.

In the above-described aspect, the calculated confidence may be increased with a decrease in a proportion of the blood-region information with respect to the fat-region information, and may be decreased with an increase in the proportion.

In the above-described aspect, the display-form manipulating portion may display the fat region indicated by the fat-region information for which the calculated confidence is lower than the reference confidence in an emphasized manner as compared with the peripheral region. The display-form manipulating portion may display the peripheral region in an emphasized manner as compared with the fat region indicated by the fat-region information for which the calculated confidence is lower than the reference confidence.

In the above-described aspect, the display-form manipulating portion may notify a surgeon about the fat region indicated by the fat-region information for which the calculated confidence is lower than the reference confidence.

A second aspect of the present invention is a biological observation apparatus including: an irradiating portion that can radiate illumination light onto biological tissue; an image-acquisition portion that captures, of reflected light, which is the illumination light radiated from the irradiating portion and reflected at the biological tissue, reflected light in a specific wavelength band, thus acquiring the biological-tissue image; any of the above-described image-processing apparatuses that process the biological-tissue image acquired by the image-acquisition portion; and a display portion that displays the biological-tissue image processed by the image-processing apparatus.

The above-described aspect may be provided with a control portion that causes white light to be emitted as illumination light to be radiated onto the biological tissue by the irradiating portion in the case in which the calculated confidence is lower than the reference confidence and that causes the image-acquisition portion to capture reflected light of the white light itself reflected at the biological tissue.

A third aspect of the present invention is an image-processing method including: a fat-region-information detecting step of detecting fat-region information that indicates a fat region in which fat exists in a biological-tissue image; a blood-region-information detecting step of detecting blood-region information that indicates a blood region in which blood exists in the biological-tissue image; a confidence calculating step of calculating a confidence for the fat-region information on a basis of the fat-region-information detected in the fat-region-information detecting step and the blood-region-information detected by the blood-region-information detecting step; and a display-form manipulating step of manipulating the fat region indicated by the fat-region information for which the calculated confidence calculated in the confidence calculating step is lower than the reference confidence so as to have a display form that can be distinguished from a peripheral region.

### BRIEF DESCRIPTION OF DRAWINGS

FIG. 1 is an overall configuration diagram schematically showing a biological observation apparatus according to a first embodiment of the present invention.

FIG. 2A is a diagram showing the absorption characteristics of β-carotene and the absorption characteristics of hemoglobin.

FIG. 2B is a diagram showing the transmittance characteristics of a color filter provided in a color CCD in the biological observation apparatus in FIG. 1.

FIG. 2C is a diagram showing the light intensity characteristics of a xenon lamp in the biological observation apparatus in FIG. 1.

FIG. 2D is a diagram showing the transmittance characteristics of a filter used in a special-light observation mode of the biological observation apparatus in FIG. 1.

FIG. 3 is a block diagram showing an image-processing portion provided in the biological observation apparatus in FIG. 1.

FIG. 4 is a block diagram showing a confidence-calculating portion in FIG. 3.

FIG. 5 is a diagram showing an example of a fat image that is divided into a plurality of local regions.

FIG. 6 is a block diagram showing a display-form setting portion in FIG. 3.

FIG. 7 is a block diagram showing a manipulating portion.

FIG. 8 is a flowchart showing an image-processing method using the biological observation apparatus in FIG. 1.

FIG. 9 is a flowchart showing, in detail, image-signal manipulating processing in the image-processing method in FIG. 8.

FIG. 10A is a diagram showing an example of an image of an observation subject site, which is obtained in a white-light observation mode.

FIG. 10B is a diagram showing an example of a pre-manipulation image of the observation subject site, which is obtained in a special-light observation mode.

FIG. 10C is a diagram showing an example of an image capturing a state in which blood exists in a fat region of the observation subject site shown in FIG. 10B.

FIG. 11A is a diagram showing an example of a display form in which, in the image shown in FIG. 10C, a fat region indicated by fat-region information for which a calculated confidence is lower than a reference confidence is manipulated so as to have a color that can be distinguished from that of a peripheral region.

FIG. 11B is a diagram showing an example of a display form in which, in the image shown in FIG. 10C, the fat region indicated by the fat-region information for which the calculated confidence is lower than the reference confidence is manipulated by surrounding the fat region with an arbitrary target color so that the fat region can be distinguished from the peripheral region.

FIG. 11C is a diagram showing an example of a display form in which, in the image shown in FIG. 10C, with resect to the fat region indicated by the fat-region information for which the calculated confidence is lower than the reference confidence, manipulation for changing the brightness of a peripheral region that is not included in the fat region is applied.

FIG. 12A is a diagram showing an example of a state in which a fat image is divided into a plurality of local regions.

FIG. 12B is a diagram showing an example of a manner in which the fat region in the fat image in FIG. 12A is reset so as to have a rectangular shape.

FIG. 13 is an overall configuration diagram schematically showing a biological observation apparatus according to a second embodiment of the present invention.

FIG. 14 is a front view showing the arrangement of individual filters in a filter turret provided in the biological observation apparatus in FIG. 13.

FIG. 15A is a diagram showing the absorption characteristics of β-carotene and the absorption characteristics of hemoglobin.

FIG. 15B is a diagram showing the transmittance characteristics of a filter in a white-light observation mode of the biological observation apparatus in FIG. 13.

FIG. 15C is a diagram showing the transmittance characteristics of the filter in a special-light observation mode of the biological observation apparatus in FIG. 13.

FIG. 16 is an overall configuration diagram schematically showing a biological observation apparatus according to a first modification of the second embodiment of the present invention.

FIG. 17A is a diagram showing the absorption characteristics of β-carotene and the absorption characteristics of hemoglobin.

FIG. 17B is a diagram showing the light intensity characteristics of an LED used in a white-light observation mode of the biological observation apparatus in FIG. 16.

FIG. 17C is a diagram showing the light intensity characteristics of an LED used in a special-light observation mode of the biological observation apparatus in FIG. 16.

FIG. 18 is an overall configuration diagram schematically showing a biological observation apparatus according to a second modification of the second embodiment of the present invention.

FIG. 19A is a diagram showing the absorption characteristics of β-carotene and the absorption characteristics of hemoglobin.

FIG. 19B is a diagram showing the spectral transmittance characteristics of a color separation prism in the biological observation apparatus in FIG. 18.

FIG. 19C is a diagram showing the light intensity characteristics of a xenon lamp in the biological observation apparatus in FIG. 18.

FIG. 19D is a diagram showing the transmittance characteristics of a filter used in a special-light observation mode of the biological observation apparatus in FIG. 18.

### DESCRIPTION OF EMBODIMENTS

### First Embodiment

An image-processing portion (image-processing apparatus), a biological observation apparatus provided with the same, and an image-processing method using these, according to a first embodiment of the present invention, will be described below with reference to the drawings.

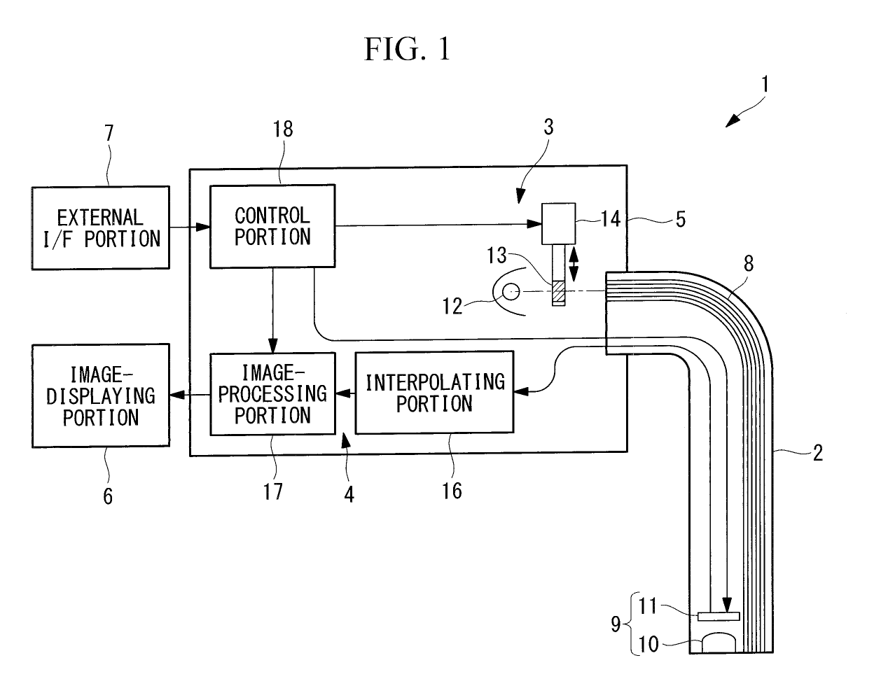

As shown in FIG. 1, a biological observation apparatus 1 according to this embodiment is an endoscope provided with: an inserted portion 2 that is inserted into a biological body; a main body portion 5 provided with a light-source portion (irradiating portion) 3 connected with the inserted portion 2 and a signal processing portion 4; an image-displaying portion (display portion) 6 that displays an image generated by the signal processing portion 4; and an external interface portion (hereinafter, referred to as “external I/F portion”) 7 with which an operator makes inputs.

The inserted portion 2 is provided with: an illumination optical system 8 with which light input from the light-source portion 3 is radiated toward an imaging subject; and an imaging optical system (image-acquisition portion) 9 that captures reflected light coming from the imaging subject.

The illumination optical system 8 is a light-guide cable that is disposed over the entire length of the inserted portion 2 in the longitudinal direction thereof and that guides the light coming from the light-source portion 3 and entering from the basal end thereof to the distal end thereof.

The imaging optical system 9 is provided with: an objective lens 10 that collects light coming from the imaging subject, which is reflected light of the light radiated onto the imaging subject by the illumination optical system 8; and an image-acquisition device 11 that captures the light collected by the objective lens 10.

The image-acquisition device 11 is, for example, a color CCD.

The light-source portion 3 is provided with a xenon lamp 12 that emits white light in a wide wavelength band; a short-wavelength cut filter 13 that can be inserted into and retracted from the optical path of the light coming from the xenon lamp 12 in order to cut out light in a predetermined wavelength from the white light emitted from the xenon lamp 12; and a linear motion mechanism 14 that is controlled by a control portion 18, described later, and that inserts the short-wavelength cut filter 13 into the optical axis and retracts it therefrom.

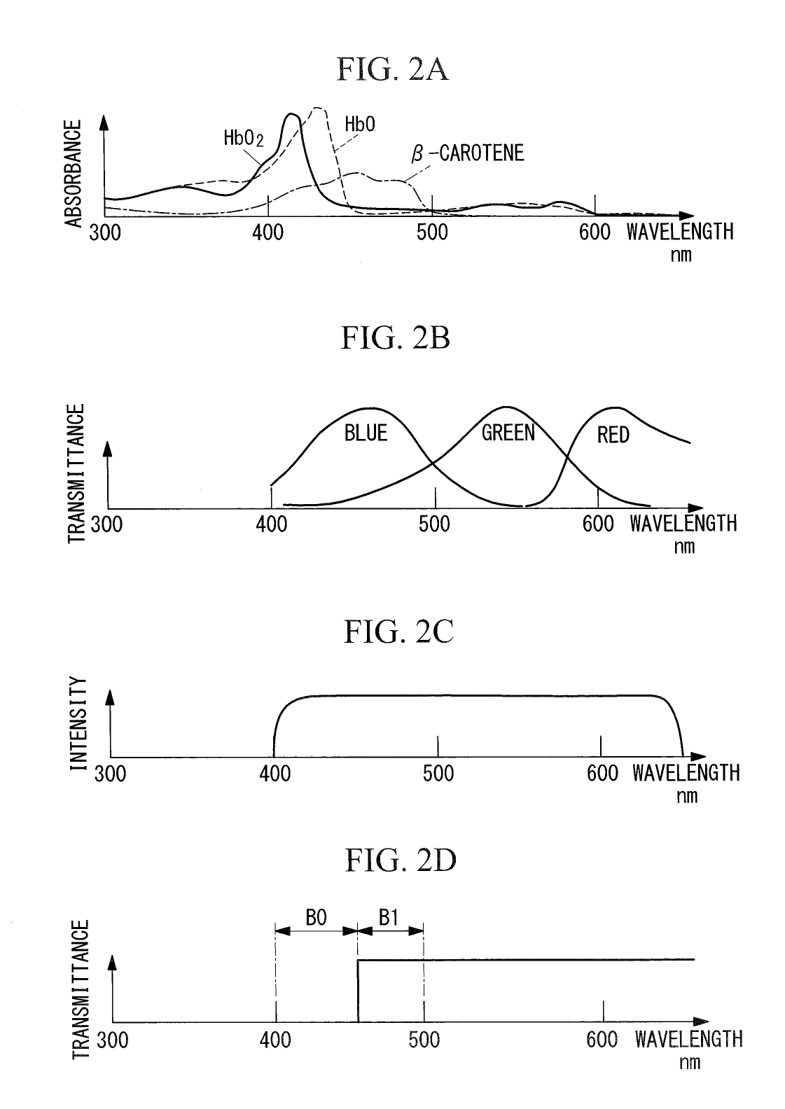

As shown in FIG. 2D, the short-wavelength cut filter 13 blocks light in a wavelength band less than 450 nm and allows light in the wavelength band equal to or greater than 450 nm to pass therethrough.

As shown in FIG. 2B, the image-acquisition device 55 is provided with a color filter (not shown) that has transmittances for separate colors.

The xenon lamp 12 has an intensity spectrum shown in FIG. 2C.

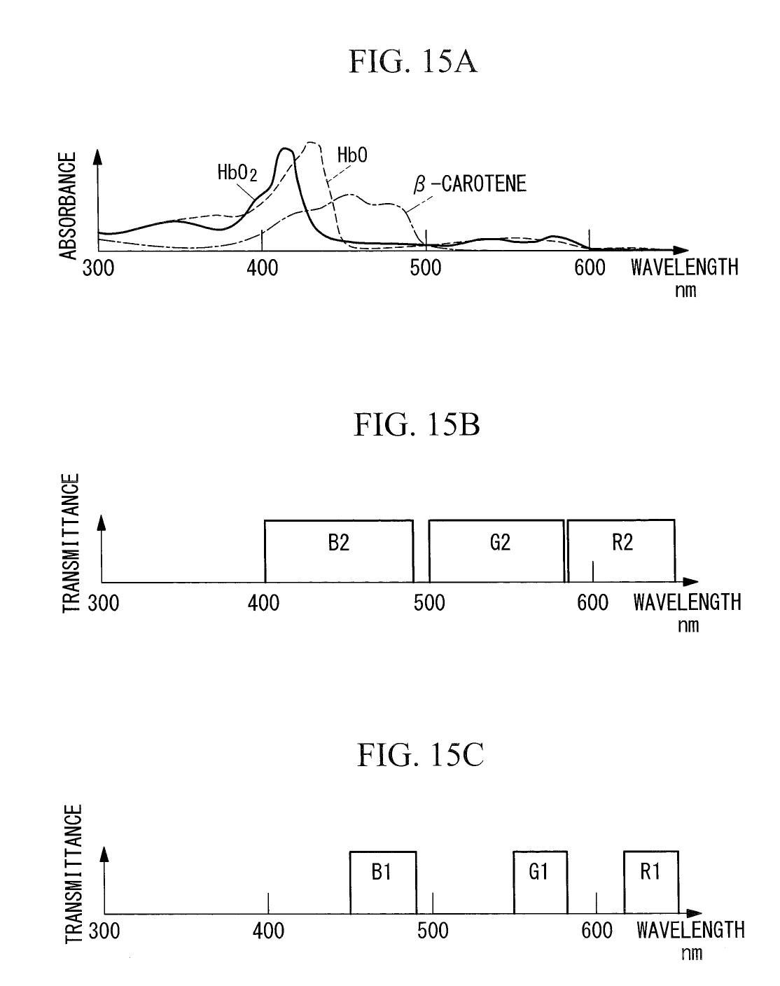

Here, as shown in FIG. 2A, β-carotene contained in biological tissue exhibits high absorption characteristics in the region of 400 to 500 nm. Hemoglobin (HbO₂, HbO), which is a component in blood, exhibits high absorption characteristics in a wavelength band equal to or less than 450 nm and a wavelength band from 500 to 600 nm. These characteristics are also applicable to FIGS. 15A, 17A, and 19A.

In other words, the wavelength band for blue in the color filter in the image-acquisition device 11 includes a wavelength band in which the absorption by hemoglobin is greater than the absorption by β-carotene and a wavelength band in which the absorption by β-carotene is greater than the absorption by hemoglobin. By inserting the short-wavelength cut filter 13 into the optical axis, only the light in the wavelength band in which the absorption by β-carotene is greater than the absorption by hemoglobin in the wavelength band for blue passes therethrough and is radiated onto the imaging subject.

Thus, in an image obtained by radiating the light in the wavelength band for blue, the influence due to the absorption by blood vessels (hemoglobin) is low, and the absorption by fat tissue (β-carotene) is high. On the other hand, by retracting the short-wavelength cut filter 13 from the optical axis, because light in the entire wavelength band for blue is radiated onto the imaging subject, it is possible to acquire a white-light image together with red and green beams that are simultaneously radiated.

Because the wavelength band for green is a region in which there is no absorption by β-carotene but there is absorption by hemoglobin, in an image obtained by radiating light in the wavelength band for green, a region in which the intensity is low is a region in which blood exists, thus indicating, for example, blood vessels.

Because the absorptions by β-carotene and hemoglobin both do not occur in the wavelength band for red, an image obtained by radiating this light is an image showing morphological features of the biological tissue surface.

The signal processing portion 4 is provided with: an interpolating portion 16 that applies demosaicing processing to the image signals (biological-tissue image) acquired by the image-acquisition device 11; an image-processing portion (image-processing apparatus) 17 that processes the image signals processed by the interpolating portion 16; and a control portion 18 that controls the image-acquisition device 11, the linear motion mechanism 14, and the image-processing portion 17.

The control portion 18 synchronizes, on the basis of instruction signals from the external I/F portion 7, the timing at which images are captured by the image-acquisition device 11, insertion and retraction of the short-wavelength cut filter 13, and the timing at which image processing is performed by the image-processing portion 17. The control portion 18 stores an OB clamp value, a gain correction value, a WB coefficient value, a gradation conversion coefficient, a color conversion coefficient, an outline emphasis coefficient, and so forth that are used in the image processing performed by the image-processing portion 17.

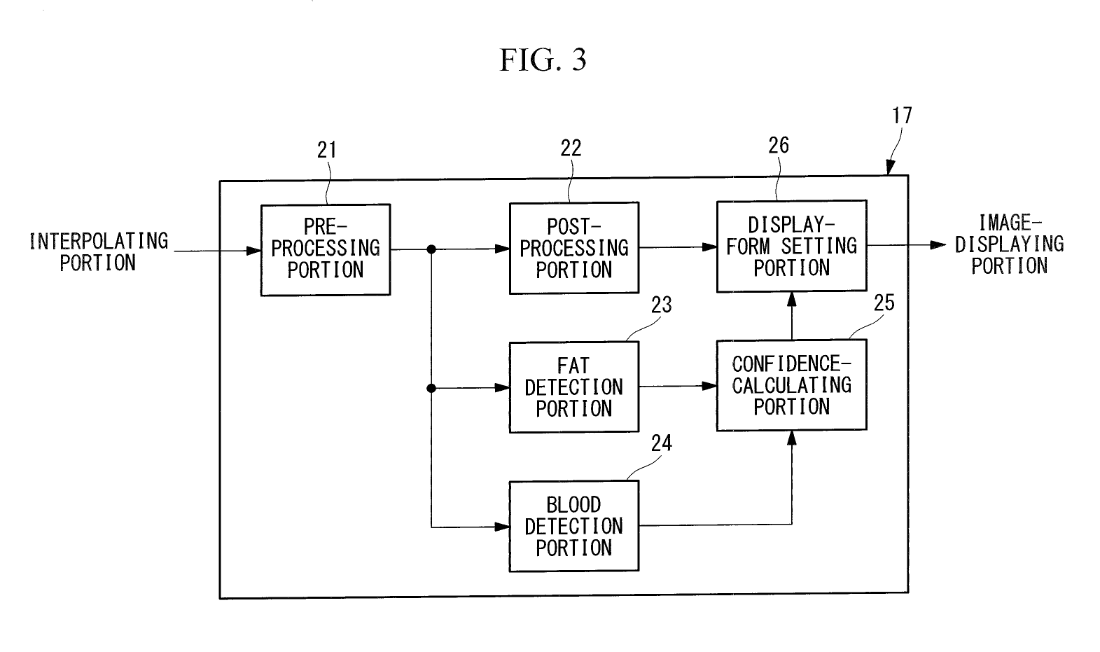

As shown in FIG. 3, the image-processing portion 17 is provided with a pre-processing portion 21; a post-processing portion 22; a fat detection portion 23; a blood detection portion 24; a confidence-calculating portion 25; and a display-form setting portion (display-form manipulating portion) 26. These components are connected to the control portion 18 and are individually controlled by the control portion 18.

The pre-processing portion 21 applies pre-processing, such as OB clamp processing, gain correction processing, and WB correction processing, to the image signals transmitted thereto from the interpolating portion 16 by using the OB clamp value, the gain correction value, and the WB coefficient value stored in the control portion 18. The pre-processing portion 21 transmits the pre-processed image signals to the post-processing portion 22, the fat detection portion 23, and the blood detection portion 24.

The post-processing portion 22 applies post-processing, such as gradation conversion processing, color processing, and outline emphasis processing, to the pre-processed image signals transmitted thereto from the pre-processing portion 21 by using the gradation conversion coefficient, the color conversion coefficient, and the outline emphasis coefficient stored in the control portion 18, thus generating a color image to be displayed on the image-displaying portion 6. The post-processing portion 22 transmits the post-processed image signals to the display-form setting portion 26.

The fat detection portion 23 generates fat image signals on the basis of the pre-processed image signals transmitted thereto from the pre-processing portion 21. The pre-processed image signals include image signals that correspond to three types of, that is, blue, green, and red, illumination light beams. The fat detection portion 23 generates one-channel fat image signals from these three types (three channels) of image signals. The fat image signals take greater signal values with an increase in the amount of β-carotene contained in the imaging subject. The fat detection portion 23 transmits the generated fat image signals to the confidence-calculating portion 25.

The blood detection portion 24 generates blood image signals on the basis of the pre-processed image signals transmitted thereto from the pre-processing portion 21. As described above, the pre-processed image signals include the image signals that correspond to the three types of, that is blue, green, and red, illumination light beams, and the blood detection portion 24 generates one-channel blood image signals from two types (two channels) of, that is, green and red, image signals. The blood image signals take greater signal values with an increase in the amount of hemoglobin contained in the imaging subject. The blood detection portion 24 transmits the generated blood image signals to the confidence-calculating portion 25.

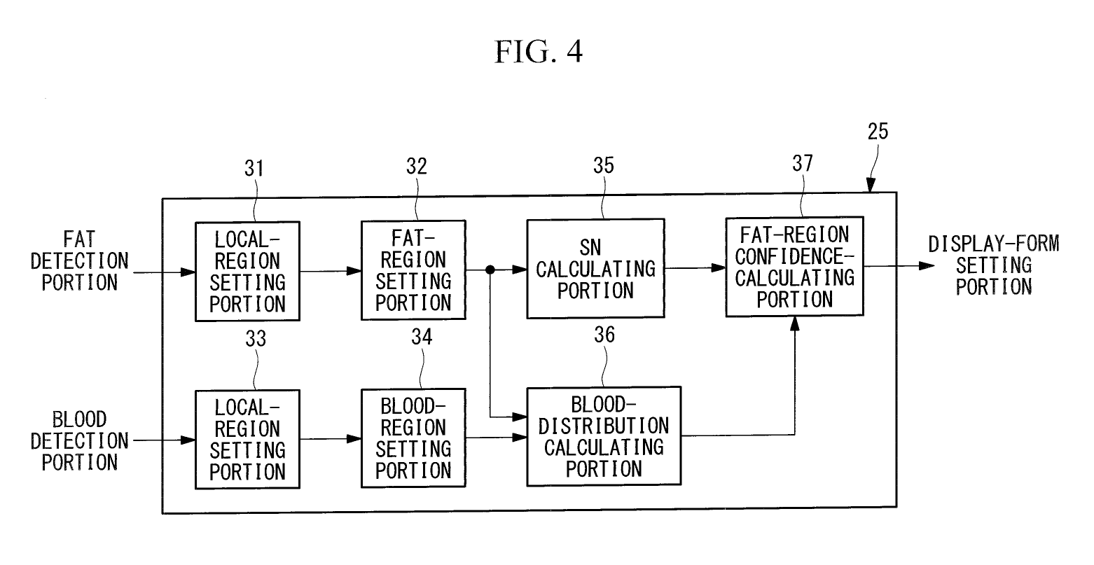

As shown in FIG. 4, the confidence-calculating portion 25 is provided with: a local-region setting portion 31; a fat-region setting portion (fat-region-information detecting portion) 32; a local-region setting portion 33; a blood-region setting portion (blood-region-information detecting portion) 34; an SN calculating portion 35; a blood-distribution calculating portion 36; and a fat-region confidence-calculating portion (confidence-calculating portion) 37. These components are connected to the control portion 18, and are individually controlled by the control portion 18.

The local-region setting portion 31 sets a plurality of local regions (blocks in a narrow sense) with respect to the fat image signals transmitted thereto from the fat detection portion 23. For example, the local-region setting portion 31 divides a fat image into rectangular regions, and sets the individual divided regions as local regions.

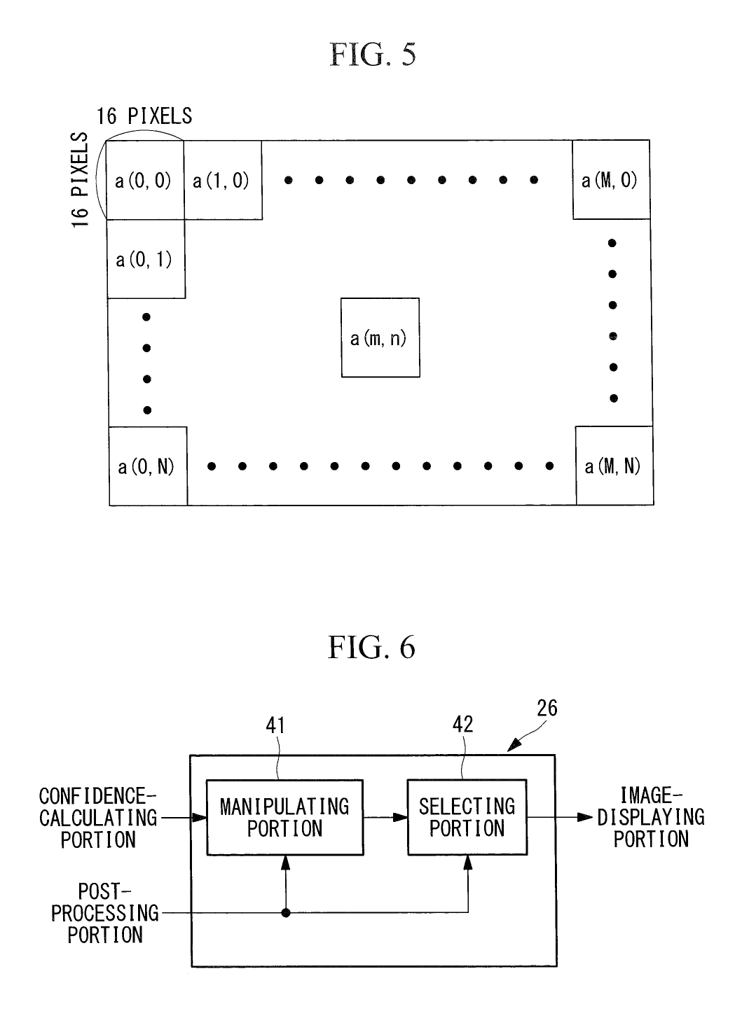

Although it is possible to appropriately set the sizes of the rectangular regions, in this embodiment, for example, one local region is assumed to be a 16×16 pixel region, as shown in FIG. 5. A fat image is assumed to be formed of M×N local regions, and the coordinates of the individual local regions are indicated by using a notation (m, n). A local region at coordinates (m, n) is denoted as a(m, n). In FIG. 5, the coordinates of a local region positioned at the upper left corner of an image are assumed to be (0, 0), the right direction is assumed to be the positive direction of m, and the downward direction is assumed to be the positive direction of n.

The local regions need not be rectangular, and it is needless to say that a fat image can be divided into arbitrary polygons, and the individual divided regions can be set as local regions. A configuration in which local regions can be arbitrarily set in accordance with instructions from the operator may be employed. In this embodiment, although one local region is defined as a region formed of a plurality of adjacent pixel groups in order to reduce the amount of subsequent calculation and to remove noise, it is possible to set one local region so as to be formed of one pixel. There is no difference in terms of subsequent processing to be performed in this case.

The fat-region setting portion 32 sets fat regions in which fat exists in the fat image. In this embodiment, as the fat regions, the fat-region setting portion 32 sets regions in which the amount of β-carotene is high. Specifically, first, the fat-region setting portion 32 applies threshold processing to all of the local regions set by the local-region setting portion 31, and identifies the local regions for which the values of the fat image signals are sufficiently high.

The fat-region setting portion 32 performs processing for integrating, among the identified local regions, the adjacent local regions with each other, and sets the individual regions obtained as a result of the integration processing as the fat regions. In the case in which there is one local region, the region is set to be the fat region. The fat-region setting portion 32 calculates the positions of all pixels included in the fat regions on the basis of the coordinates of the local regions a(m, n) included in the fat regions and information about pixels included in the individual local regions, and transmits the calculation results to the SN calculating portion 35 and the blood-distribution calculating portion 36 as fat-region information indicating the fat regions.

The local-region setting portion 33 sets a plurality of local regions (blocks in a narrow sense) with respect to the blood image signals transmitted thereto from the blood detection portion 24. Because the manner in which the local regions are set by the local-region setting portion 33 is similar to the manner in which the local regions are set by the local-region setting portion 31, the description thereof will be omitted.

The blood-region setting portion 34 sets blood regions in which blood exists in the blood image. In this embodiment, the blood-region setting portion 34 sets regions in which the amount of hemoglobin is high as the blood regions. The manner in which the blood regions are set by the blood-region setting portion 34 is similar to the manner in which the fat regions are set by the fat-region setting portion 32.

In other words, the blood-region setting portion 34 applies threshold processing to all of the local regions set by the local-region setting portion 33, identifies the local regions for which the values of the blood image signals are sufficiently high, applies the processing for integrating the adjacent local regions with each other, and sets the thus-obtained individual regions as the blood regions. The blood-region setting portion 34 calculates positions of all of pixels included in the blood regions on the basis of the coordinates of the local regions a(m, n) included in the blood regions and information about pixels included in the individual local regions, and transmits the calculation results to the blood-distribution calculating portion 36 as blood-region information indicating the blood regions.

The SN calculating portion 35 calculates an SN ratio of the fat-region information transmitted thereto from the fat-region setting portion 32. For example, as the SN ratio, a ratio between the signal levels and noise of the fat-region information may be determined. Specifically, the SN calculating portion 35 calculates an average (Ave) of the signal levels of the fat-region information, sufficiently reduces noise by applying noise reduction processing to the fat-region information, and calculates differences between the fat-region information before noise reduction and the fat-region information after noise reduction. The standard deviation of these differences is calculated and used as a noise level (Noise).

The SN ratio is calculated by using expression (1) below:

SN ratio=20×log₁₀(Ave/Noise) (1).

Here, the SN ratio indicates a degree to which the detection precision with respect to the fat regions is deteriorated due to disturbances (blood, forceps, mist, or the like) during surgery, and a lower SN ratio indicates a lower confidence for the fat-region information. The SN calculating portion 35 transmits the calculated SN ratio of the fat-region information to the fat-region confidence-calculating portion 37.

The blood-distribution calculating portion 36 calculates blood-distribution signals, which indicate proportions at which the blood regions occupy the fat regions, on the basis of the fat regions indicated by the fat-region information transmitted thereto from the fat-region setting portion 32 and the blood regions indicated by the blood-region information transmitted thereto from the blood-region setting portion 34. It suffices if it is possible to ascertain, for example, the magnitude of the extent (area) to which blood exists in the fat regions.

Specifically, the blood-distribution calculating portion 36 counts the number of pixels (BkNum) in the fat regions and counts the number of pixels (HbNum) in the blood regions that exist in the fat regions, thus calculating blood-distribution signals (HbDist) by using expression (2) below:

HbDist=HbNum/BkNum (2).

Here, the blood-distribution signals indicate the degree to which blood exists in the fat regions, and the magnitude thereof is increased with a greater presence of blood. Greater blood-distribution signals indicate a lower confidence for the fat-region information. The blood-distribution calculating portion 36 transmits the calculated blood-distribution signals to the fat-region confidence-calculating portion 37.

The fat-region confidence-calculating portion 37 calculates the confidence for the fat-region information on the basis of the SN ratio of the fat-region information transmitted thereto from the SN calculating portion 35 and the blood-distribution signals transmitted thereto from the blood-distribution calculating portion 36. Specifically, the fat-region confidence-calculating portion 37 calculates a confidence (BkTrust) for the fat-region information in the form of a linear sum of the SN ratio (SN) of the fat-region information and the blood-distribution signals (HbDist) by using expression (3) below:

BkTrust=α×SN+β×(1/HbDist) (3).

Here, the confidence for the fat-region information takes a greater value with an increase in the detection precision with respect to the fat regions. α and β are constant terms and are parameters that can be adjusted depending on whether the influence of disturbances (including blood) is emphasized or the influence of (only) blood is emphasized when calculating the confidence for the fat-region information. The parameters can be set by the operator by means of the external I/F portion 7 via the control portion 18. The fat-region confidence-calculating portion 37 transmits the fat-region information and the confidence therefor to the display-form setting portion 26. In the following, the confidence for the fat-region information calculated by the fat-region confidence-calculating portion 37 will be referred to as the calculated confidence.

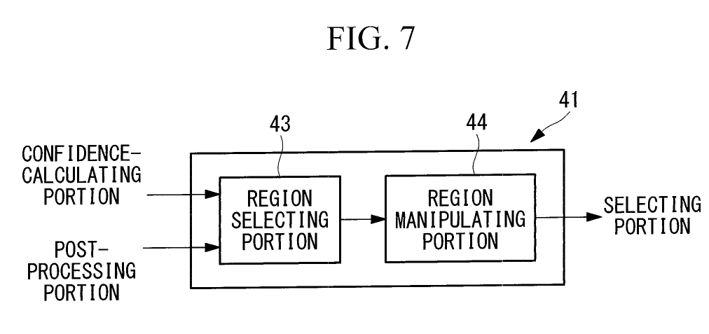

As shown in FIG. 6, the display-form setting portion 26 is provided with: a manipulating portion 41 that manipulates, on the basis of the fat-region information and the calculated confidence therefor transmitted thereto from the confidence-calculating portion 25, the post-processed image signals transmitted thereto from the post-processing portion 22; and a selecting portion 42 that selects an image to be displayed on the image-displaying portion 6. These components are connected to the control portion 18 and are individually controlled by the control portion 18.

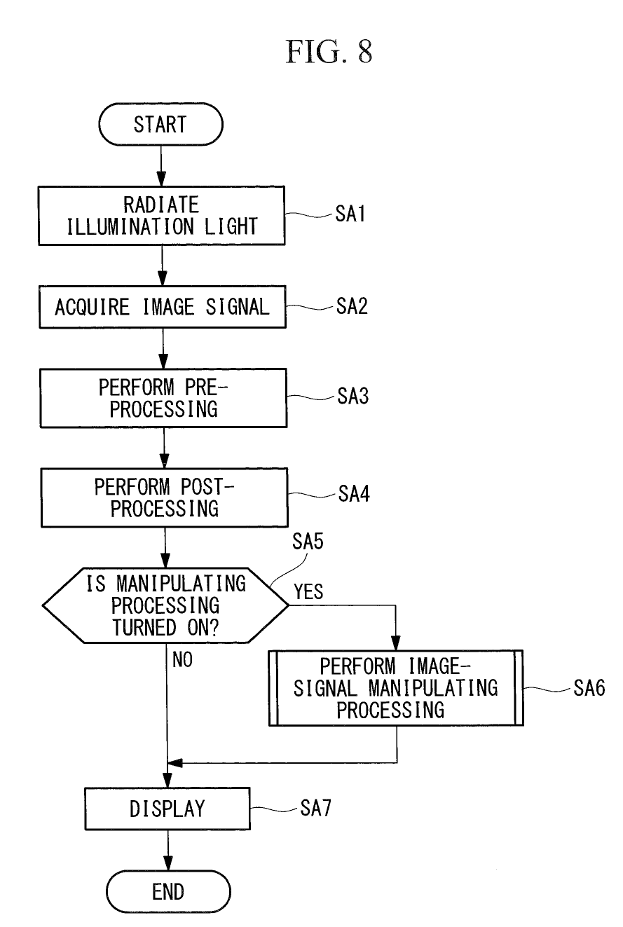

As shown in FIG. 7, the manipulating portion 41 is provided with: a region selecting portion 43; and a region manipulating portion 44.

The region selecting portion 43 selects, among the sets of the fat-region information transmitted thereto from the confidence-calculating portion 25, the fat-region information of a region of interest. Specifically, the region selecting portion 43 selects, among the sets of the fat-region information, the fat-region information for which the calculated confidence is lower than a reference confidence that is set in advance and that serves as a reference. By performing such processing, it is possible to select the fat-region information for which the confidence is low (disturbance is high) by excluding the fat-region information for which the confidence is high (disturbance is low).

Next, the region selecting portion 43 sets, in the post-processed image signals transmitted thereto from the post-processing portion 22, a region indicated by the fat-region information, selected in advance, for which the calculated confidence is lower than the reference confidence, to be a corresponding region of interest and transmits the information about pixels in the set corresponding region of interest to the region manipulating portion 44 so as to serve as corresponding region-of-interest information.

The region manipulating portion 44 performs color conversion processing by using expressions (4) to (6) below with respect to the pixels indicated by the corresponding region-of-interest information among the post-processed image signals transmitted thereto from the region selecting portion 43:

r_out(x,y)=gain×r(x,y)+(1−gain)×Tr (4);

g_out(x,y)=gain×g(x,y)+(1−gain)×T_g (5); and

b_out(x,y)=gain×b(x,y)+(1−gain)×Tb (6).

Here, r(x, y), g(x, y), and b(x, y) are signal values of R, G, and B channels at the coordinates (x, y) of the image signals before the color conversion, and r_out(x, y), g_out(x, y), and b_out(x, y) are signal values of the R, G, and B channels of the image after the color conversion. T_r, T_g, and T_b are R, G, and B signal values of an arbitrary target color, and the gain is an arbitrary coefficient from 0 to 1.

With this processing, the fat regions indicated by the fat-region information for which the calculated confidences are lower than the reference confidence are manipulated to have a color that is different as compared with that of the peripheral region. The region manipulating portion 44 transmits the manipulated image signals to the selecting portion 42. The region manipulating portion 44 may perform manipulation that assigns priority to the fat regions, for example, in ascending order of the calculated confidences for the fat-region information.

The selecting portion 42 selects one of the post-processed image signals transmitted thereto from the post-processing portion 22 and the manipulated image signals transmitted thereto from the manipulating portion 41 and transmits the selected image signals to the image-displaying portion 6. For example, in the case in which the fat-region information is not detected, the post-processed image signals transmitted from the post-processing portion 22 are selected as a display image, and, in the case in which the fat-region information is detected, the image signals that have been subjected to the manipulating processing and transmitted from the manipulating portion 41 are selected as the display image.

In the case in which it is necessary to turn ON/OFF the manipulating processing of the image signals, the operator may perform setting via the external I/F portion 7 so that control is performed on the basis of control signals input to the selecting portion 42 from the control portion 18.

The image-displaying portion 6 is a display apparatus that can display a video image and is constituted of, for example, a CRT, a liquid crystal monitor, or the like. The image-displaying portion 6 displays the image transmitted thereto from the selecting portion 42.

The external I/F portion 7 is an interface with which the operator performs inputs or the like to the endoscope apparatus. The external I/F portion 7 has a manipulating-processing button (not shown) with which it is possible to give instructions about turning ON/OFF the image-signal manipulating processing, and the operator can give, via the external I/F portion 7, the instructions about turning ON/OFF the image-signal manipulating processing. The instruction signals about turning ON/OFF the image-signal manipulating processing given via the external I/F portion 7 are output to the control portion 18. The external I/F portion 7 includes a power source switch for turning ON/OFF a power source, a mode switching button for switching between an image capturing mode and other various modes, and so forth.

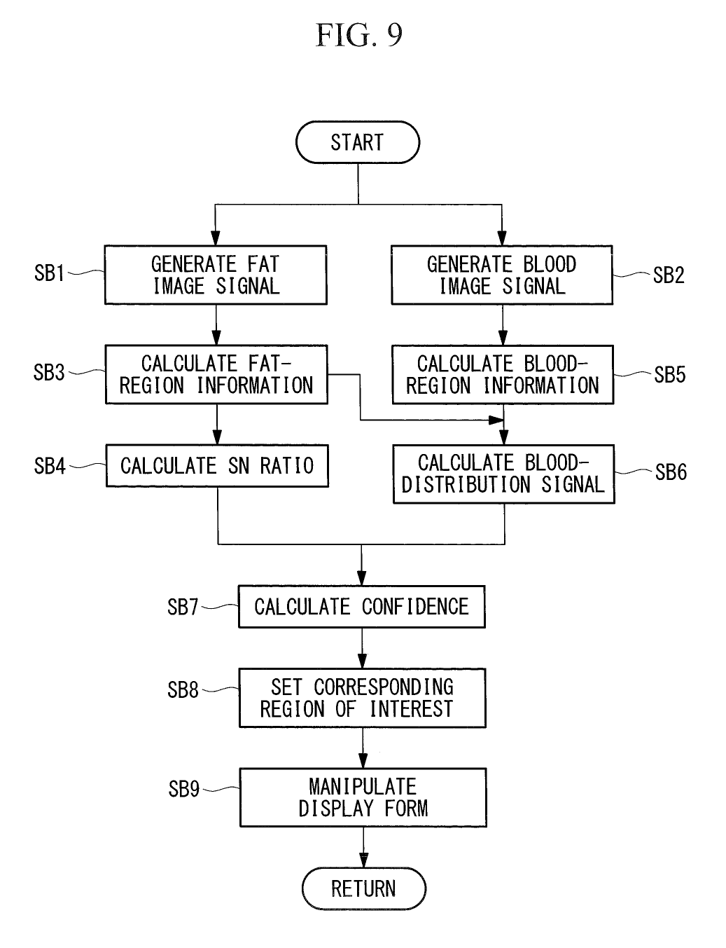

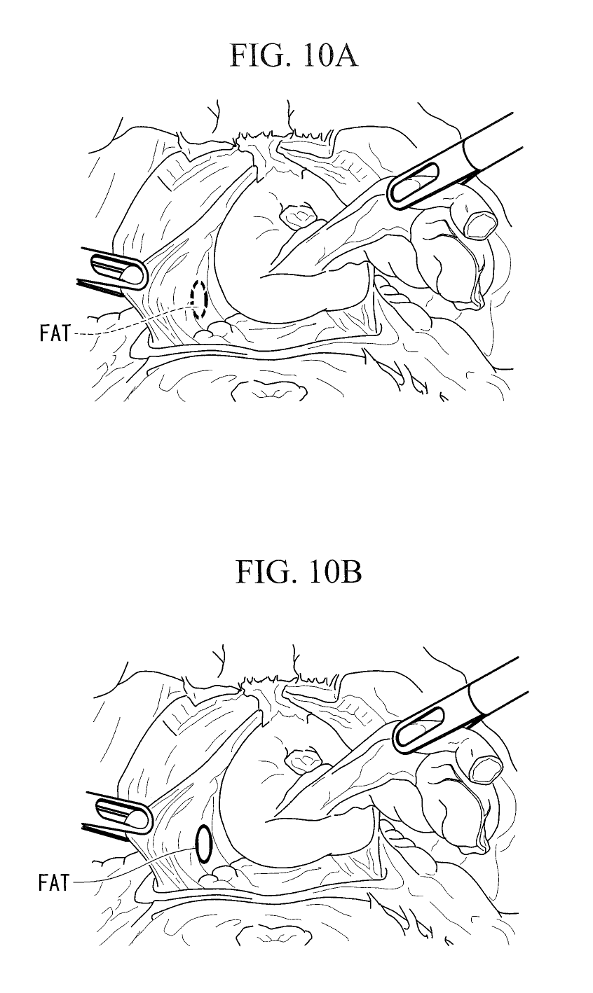

An image-processing method using the thus-configured biological observation apparatus 1 and image-processing portion 17 according to this embodiment will be described with reference to the flowcharts in FIGS. 8 and 9.

In order to observe a biological body by using the biological observation apparatus 1 according to this embodiment, first, the inserted portion 2 is inserted into the body cavity, and the distal end of the inserted portion 2 is made to face the observation subject site. The operator sets, via the external I/F portion 7, the instruction signals for turning ON/OFF the image-signal manipulating processing to OFF, which causes the control portion 18 to actuate the linear motion mechanism 14, thus retracting the short-wavelength cut filter 13 from the optical axis.

Subsequently, as shown in FIG. 8, the white light in the wide wavelength band emitted from the xenon lamp 12 is guided to the distal end of the inserted portion 2 via the light-guide cable 7, and the respective illumination light beams are radiated onto the observation subject site (imaging subject) (illumination-light radiating step SA1). The white light radiated onto the observation subject site is reflected at a surface of the observation subject site and is subsequently collected by the objective lens 10, thus being captured by the image-acquisition device 11 (image-signal acquiring step SA2).

Because the image-acquisition device 11, which is constituted of the color CCD, is provided with the color filter having transmittances for separate colors, the pixels corresponding to the respective colors individually acquire image signals. The image signals acquired by the image-acquisition device 11 are subjected to the demosaicing processing by the interpolating portion 16, and are transmitted to the image-processing portion 17 after being converted to the three-channel image signals.

In the image-processing portion 17, the image signals transmitted thereto from the interpolating portion 16 are subjected, by the pre-processing portion 21, to pre-processing such as the OB clamp processing, the gain correction processing, and the WB correction processing by using the OB clamp value, the gain correction value, and the WB coefficient value stored in the control portion 18 (pre-processing step SA3), and are transmitted to the post-processing portion 22.

Next, the pre-processed image signals transmitted from the pre-processing portion 21 are subjected, by the post-processing portion 22, to post-processing such as the gradation conversion processing, the color processing, and the outline emphasis processing by using the gradation conversion coefficient, the color conversion coefficient, and the outline emphasis coefficient stored in the control portion 18, and thus the white-light image to be displayed on the image-displaying portion 6 is generated (post-processing step SA4).

Subsequently, the control portion 18 determines the instruction signals for turning ON/OFF the image-signal manipulating processing transmitted thereto from the external I/F portion 7 (manipulating processing determining step SA5). Because the instruction signals for turning ON/OFF the image-signal manipulating processing are set to OFF, the white-light image generated by the post-processing portion 22 is displayed on the image-displaying portion 6 via the display-form setting portion 26 (displaying step SA7). This observation mode will be referred to as the white-light observation mode.

In the white-light observation mode, the operator can observe the morphology of the biological tissue by using the white-light image displayed on the image-displaying portion 6. In the white-light image, for example, in a region in which blood vessels exist, because absorptions occur in wavelength bands for blue B2 and green G2, the blood vessels are displayed in red. In a region in which fat exists, because absorption occurs in blue B2, fat is displayed in yellow.

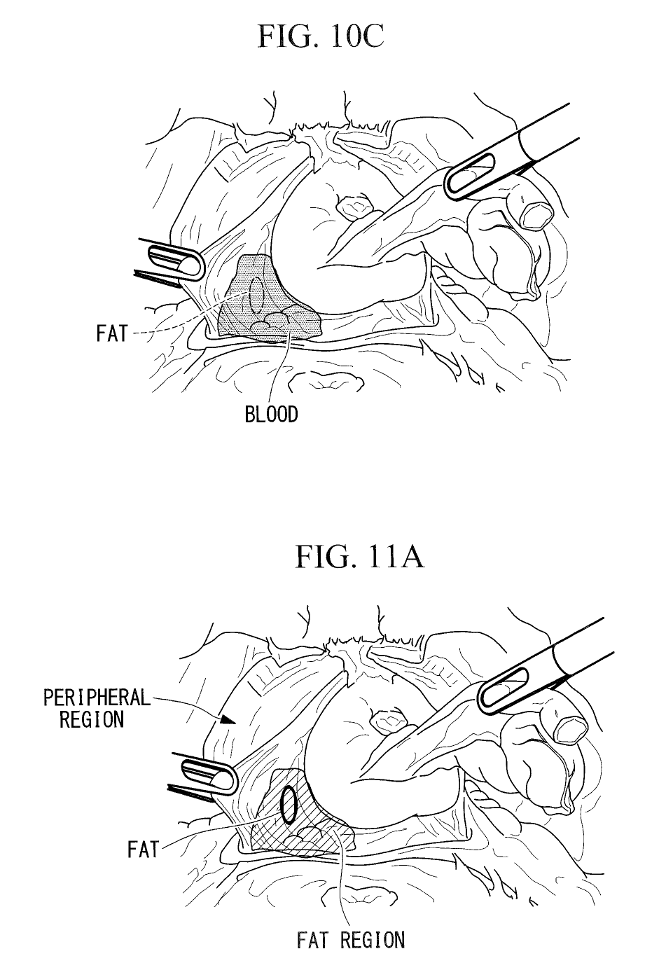

However, with the white-light image, in the case in which fat tissue is extremely thin, the color of blood vessels in an organ behind the fat tissue passes therethrough, thus making it difficult to ascertain the presence of the fat tissue. For example, FIG. 10A shows an image of an observation subject site obtained in the white-light observation mode, and, although the image is bright as a whole and is highly visible, it is difficult to visually ascertain fat that exists in a fascia.

Therefore, in such a case, the operator switches, via the external I/F portion 7, the instruction signals for turning ON/OFF the image-signal manipulating processing to ON, which causes the control portion 18 to actuate the linear motion mechanism 14, thus inserting the short-wavelength cut filter 13 into the optical axis of the light coming from the xenon lamp 12.

The white light emitted from the xenon lamp 12 passes through the short-wavelength cut filter 13 that cuts the light therein in the wavelength band that is equal to or less than 450 nm, and is radiated onto the observation subject site from the distal end of the inserted portion 2 via the light-guide cable 7 (illumination-light radiating step SA1). The reflected light, which is reflected at the surface of the observation subject site when the white light is radiated thereto, is collected by the objective lens 9, and is captured by the image-acquisition device 11 (image-signal acquiring step SA2).

Although the image signals acquired by the pixels in the image-acquisition device 11 corresponding to green and red are not different from the case of the white-light observation mode, with the image signals acquired by the pixels corresponding to blue, the wavelength band that is equal to or less than 450 nm is cut out therefrom, and thus, the image signals are converted to signals in a wavelength band from 450 to 500 nm. The image signals acquired by the image-acquisition device 11 are subjected to the demosaicing processing by the interpolating portion 16, are converted to the three-channel image signals, and are subsequently transmitted to the image-processing portion 17.

The wavelength band B1 from 450 to 500 nm, which is blue, in a special-light observation mode is a wavelength band in which the absorption by R-carotene is greater than the absorption by hemoglobin as compared with a wavelength band B0 from 400 to 450 nm, which has been cut out by the short-wavelength cut filter 13. Therefore, in an image obtained by radiating light in this wavelength band B1, the influence of the absorption by blood is lower and the influence of the absorption by fat is greater as compared with an image obtained by radiating light in the wavelength band B0. In other words, it is possible to obtain an image in which the distribution of fat is better reflected.

The wavelength band for green is a wavelength band in which the absorption by R-carotene is extremely low and the absorption by hemoglobin is high. Therefore, a region having a low luminance in an image obtained by radiating light in the wavelength band for green shows a region in which blood exists regardless of the presence of fat. In other words, it is possible to clearly display tissue that contains a large amount of hemoglobin, such as blood, blood vessels, or the like.

The wavelength band for red is a wavelength band in which the absorptions by R-carotene and hemoglobin are extremely low. Therefore, an image obtained by radiating light in the wavelength band for red shows a luminance distribution based on the shape (depressions/protrusions, a lumen, a fold, or the like) of the imaging subject.

In the image-processing portion 17, the image signals transmitted thereto from the interpolating portion 16 are pre-processed by the pre-processing portion 21 (pre-processing step SA3) and are transmitted to the post-processing portion 22, the fat detection portion 23, and the blood detection portion 24.

Next, the pre-processed image signals transmitted from the pre-processing portion 21 are post-processed by the post-processing portion 22 (post-processing step SA4), and are transmitted to the display-form setting portion 26.

Subsequently, the control portion 18 determines the instruction signals for turning ON/OFF the processed-fat emphasizing processing (manipulating processing determining step SA5), and, because the instruction signals for turning ON/OFF the image-signal manipulating processing are set to ON, the manipulating processing of the image signals is executed (image-signal manipulating processing step SA6).

As shown in FIG. 9, in the manipulating processing of the image signals, the fat detection portion 23 generates one-channel fat image signals in which the signal values are increased with an increase in the amount of R-carotene contained in the imaging subject on the basis of the three types (three channels) of, that is, blue, green, and red, image signals transmitted thereto from the pre-processing portion 21 (fat-image-signal generating step SB1), and the generated image signals are transmitted to the confidence-calculating portion 25.

The blood detection portion 24 generates one-channel blood image signals in which the signal values are increased with an increase in the amount of hemoglobin contained in the imaging subject on the basis of the two types (two channels) of, that is, green and red, image signals among the image signals transmitted thereto from the pre-processing portion 21 (blood-image-signal generating step SB2), and the generated image signals are transmitted to the confidence-calculating portion 25.

Next, in the confidence-calculating portion 25, the local-region setting portion 31 and the fat-region setting portion 32 set fat regions in the fat image signals transmitted thereto from the fat detection portion 23, and the fat-region information indicating the fat regions is calculated (fat-region-information detecting step SB3). The calculated fat-region information is transmitted to the SN-ratio calculating portion 35 and the blood-distribution calculating portion 36. Then, the SN calculating portion 35 calculates the SN ratio of the fat-region information (SN-ratio calculating step SB4).

The local-region setting portion 33 and the fat-region setting portion 32 set the blood regions in the blood image signals transmitted thereto from the blood detection portion 24, and the blood-region information indicating the blood regions is calculated (blood-region-information detecting step SB5). The calculated blood-region information is transmitted to the blood-distribution calculating portion 36. The blood-distribution calculating portion 36 calculates, on the basis of the fat-region information and the blood-region information, the blood-distribution signals that indicate the proportions at which the blood regions occupy the fat regions (blood-distribution-signal calculating step SB6).

Next, the fat-region confidence-calculating portion 37 calculates, on the basis of the SN ratio of the fat-region information and the blood-distribution signals, the confidence for the fat-region information (confidence calculating step SB7), and the calculated confidence for the calculated fat-region information is transmitted to the display-form setting portion 26.

In the display-form setting portion 26, the region selecting portion 43 sets, in the post-processed image signals transmitted thereto from the post-processing portion 22, the corresponding region of interest indicated by the fat-region information for which the calculated confidence is lower than the reference confidence (corresponding region-of-interest setting step SB8), and the corresponding region-of-interest information that indicates the pixels in the corresponding region of interest is transmitted to the region manipulating portion 44.

Next, the region manipulating portion 44 manipulates the fat regions indicated by the corresponding region-of-interest information among the post-processed image signals transmitted thereto from the post-processing portion 22 so as to have a color that is different as compared with that of the peripheral regions (display-form manipulating step SB9). The selecting portion 42 selects, as the display image, the image signals that have been subjected to the manipulating processing and that are transmitted from the manipulating portion 41, and thus, the image-displaying portion 6 displays the display image (displaying step SA7 in FIG. 8). This observation mode will be referred to as the special-light observation mode.

In the special-light observation mode, for example, as shown in FIG. 10B, with the image that has been post-processed by the post-processing portion 22, it is possible to increase the visibility of fat as compared with the image obtained in the white-light observation mode shown in FIG. 10A. However, as shown in FIG. 10C, the visibility of fat is hindered by disturbances during surgery, representative of which is blood or the like, and thus, it is impossible to accurately detect fat in some cases.

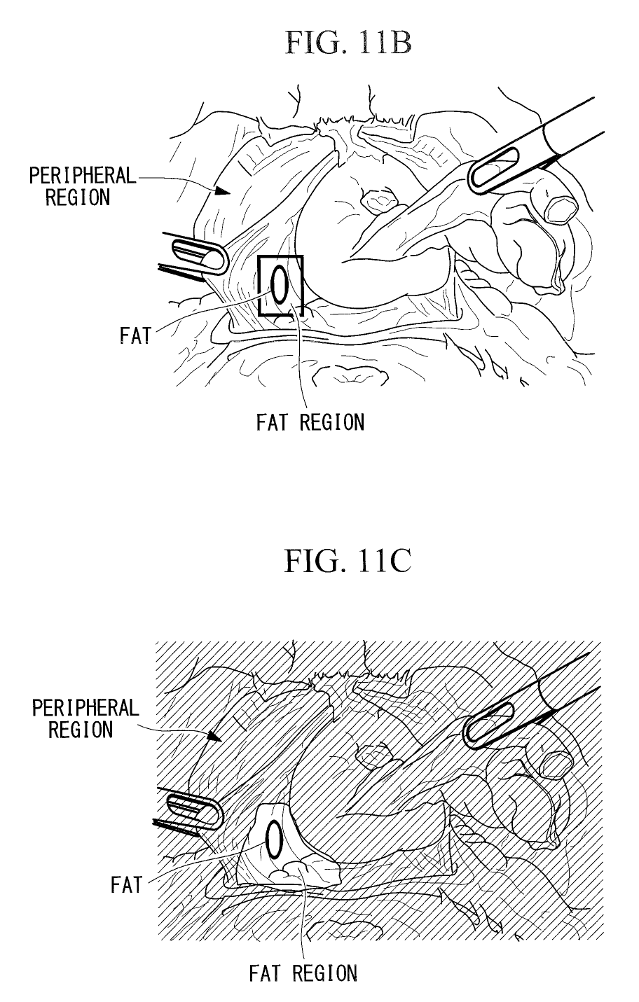

In contrast, in this embodiment, because the fat regions indicated by the fat-region information for which the calculated confidence is lower than the reference confidence are displayed after the color thereof is manipulated by the manipulating portion 41 so as to be a color that can be distinguished from that of the peripheral regions, as shown in FIG. 11A, the operator can easily ascertain the fat regions indicated by the fat-region information that cannot be accurately detected due to the influence of blood and disturbances, and he/she can perform treatment such as removing blood and disturbance in those fat regions.

In the case in which the fat-region information is not detected in the fat image signals, the selecting portion 42 selects, as the display image, the post-processed image signals transmitted thereto from the post-processing portion 22, and the image-displaying portion 6 displays the display image.

As has been described above, with the biological observation apparatus 1 and the image-processing portion 17 according to this embodiment, in the case in which, during surgery, fat-region information cannot be accurately detected because fat detection is hindered by blood existing on the imaging subject and the influence of disturbances, such as an insufficient exposure level, bright spots, mist, forceps, or the like, by manipulating the fat regions indicated by the fat-region information for which the calculated confidence is lower than the reference confidence in the biological-tissue image so as to have a display form that can be distinguished from the peripheral regions, it is possible to allow the operator to ascertain the fat regions that cannot be accurately detected because fat detection is hindered. By doing so, the operator can perform treatment such as removing blood and disturbances in those fat regions, thus reducing the risk of damaging the nerves.

In this embodiment, as shown in FIG. 11A, the region manipulating portion 44 manipulates the fat regions indicated by the fat-region information for which the calculated confidence is lower than the reference confidence so as to have a color that is different as compared with that of the peripheral region. Alternatively, for example, the region manipulating portion 44 may apply color conversion processing to all pixels that form a boundary of a corresponding region of interest indicated by the corresponding region-of-interest information in the post-processed image signals by using expressions (7) to (9) below:

r_out(x,y)=T_r (7);

g_out(x,y)=T_g (8); and

b_out(x,y)=T_b (9).

By performing such processing, as shown in FIG. 11B, it is possible to surround the fat regions indicated by the fat-region information for which the calculated confidence is lower than the reference confidence by using an arbitrary target color, thus displaying the fat regions in a form that can be distinguished as compared with that of the peripheral region.

The region manipulating portion 44 may apply luminance conversion processing to pixels in the peripheral regions that are not included in the fat regions indicated by the corresponding region-of-interest information in the post-processed image signals, as in expressions (10) to (12) below:

r_out(x,y)=gain×r(x,y) (10);

g_out(x,y)=gain×g(x,y) (11); and

b_out(x,y)=gain×b(x,y) (12).

By performing such processing, as shown in FIG. 11C, it is possible to darken the peripheral regions that are not included in the fat regions indicated by the fat-region information for which the calculated confidence is lower than the reference confidence, thus relatively increasing the visibility of the fat regions.

In this embodiment, although the region selecting portion 43 sets, as the corresponding region of interest, the fat regions indicated by the fat-region information for which the calculated confidence in the post-processed image signals is lower than the reference confidence, alternatively, the fat regions indicated by the fat-region information for which the calculated confidence in the post-processed image signals is equal to or greater than the reference confidence may be set as the corresponding region of interest.

By manipulating the fat regions indicated by the fat-region information for which the calculated confidence is equal to or greater than the reference confidence, it is possible to enhance the visibility of the fat regions indicated by the fat-region information for which the confidence is relatively lower than the reference confidence, and thus, it is possible to allow the surgeon to ascertain the fat regions that cannot be accurately detected due to the influence of blood and disturbances.

In this embodiment, although the fat-region setting portion 32 simply integrates adjacent local regions with each other among the identified local regions, alternatively, the fat-region setting portion 32 may reset the fat-region information so that the fat-region information indicates an arbitrary shape such as a polygon, a circle, or the like.

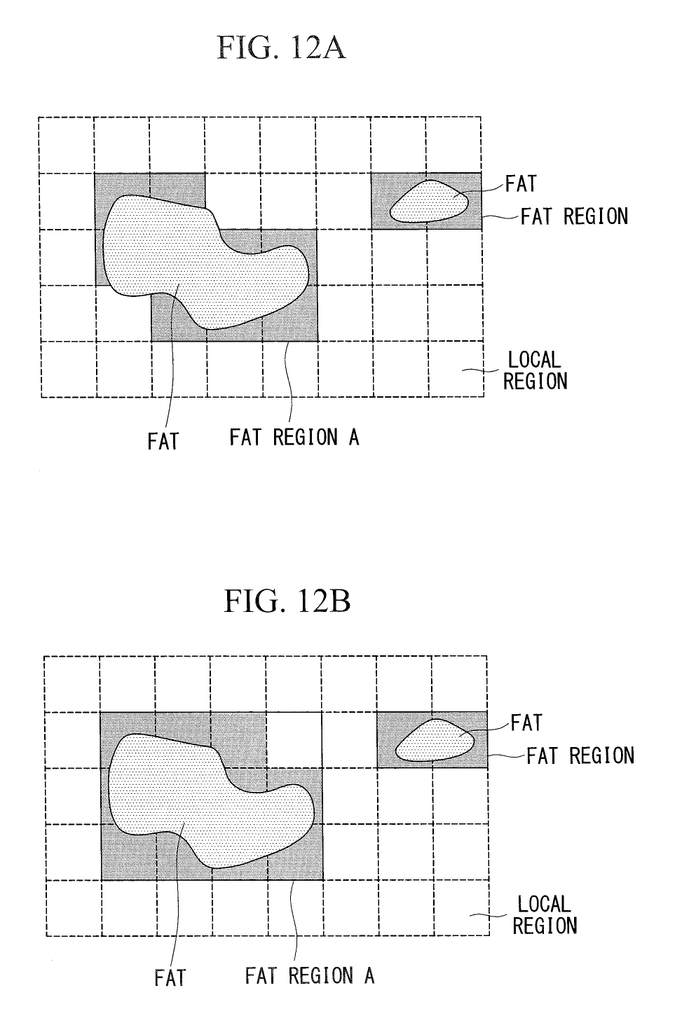

For example, FIGS. 12A and 12B show examples of fat images in which each of the regions surrounded by broken lines represents the local region. In FIG. 12A, in the case in which the shape of a fat region A needs to be set to be a quadrangle, first, positions of all of the pixels included in the fat region A are calculated on the basis of the coordinates of the local regions a(m, n) belonging to the fat region A and information about the pixels included in the individual local regions.

As shown in FIG. 12B, the quadrangles that circumscribe the collection of all of the calculated pixels may be set as the fat region A again, and the positions of all of the pixels included in the set quadrangle fat region A may be calculated and output so as to serve as the fat-region information indicating the fat region A.

By doing so, it is possible to reset the fat region A so as to have a shape that has a greater visibility.

### Second Embodiment

Next, an image-processing portion (image-processing apparatus), a biological observation apparatus provided with the same, and an image-processing method according to a second embodiment of the present invention will be described below with reference to the drawings.

In describing this embodiment, portions that have common configurations with the image-processing portion 17, the biological observation apparatus 1, and the image-processing method according to the above-described first embodiment will be given the same reference signs, and descriptions thereof will be omitted.

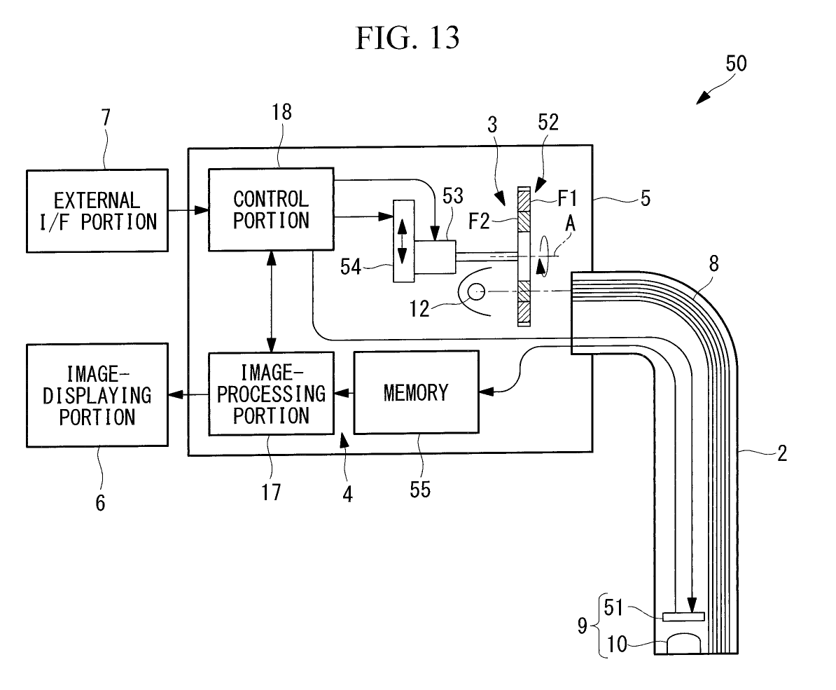

In the first embodiment, a color CCD is employed as the image-acquisition device 11, and the image signals for the three channels are simultaneously acquired. Alternatively, as shown in FIG. 13, a biological observation apparatus 50 according to this embodiment employs a monochromatic CCD as an image-acquisition device 51 and is provided with, instead of the short-wavelength cut filter 13 and the linear motion mechanism 14: a filter turret 52 that extracts light of a predetermined wavelength from the white light emitted from the xenon lamp 12 and makes the extracted light pass therethrough in a time division manner; a motor 53 that drives the filter turret 52; and a linear motion mechanism 54 that moves the filter turret 52 in a direction that intersects the optical axis of the xenon lamp 12. The signal processing portion 4 is provided with, instead of the interpolating portion 6, a memory 55 that stores the image signals acquired by the image-acquisition device 51 separately for wavelengths of the illumination light beams radiated onto the observation subject site.

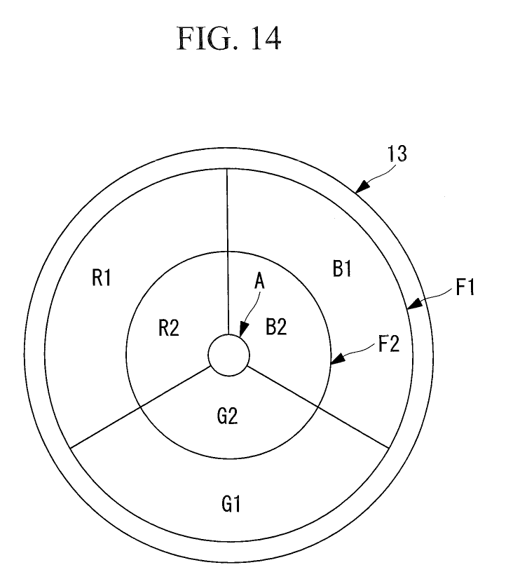

As shown in FIG. 14, the filter turret 52 is provided with, for example, two types of filter groups F1 and F2 that are concentrically disposed in a radial direction centered on a rotation center A. By disposing one of the filter groups F1 and F2 on the optical axis of the white light coming from the xenon lamp 12, the filter turret 52 can emit the light selected by the filter group F1 or F2 toward the inserted portion 2.

As shown in FIG. 15C, the first filter group F1 is configured by arraying, in a circumferential direction, filters B1, G1, and R1 that have high transmittances for blue (B1: 450 to 480 nm), green (G1: 550 to 570 nm), and red (R1: 620 to 650 nm) among the wavelength bands for blue, green, and red.

As shown in FIG. 15B, the second filter group F1 is configured by arraying, in a circumferential direction, filters B2, G2, and R2 that individually allow light in nearly continuous wavelength bands, that is, blue (B2: 400 to 490 nm), green (G2: 500 to 570 nm), and red (R2: 590 to 650 nm), to pass therethrough. FIGS. 15A and 2A show the same graph.

Because the wavelength band for blue in the first filter group F1 is a wavelength band in which the absorption by β-carotene is greater than the absorption by hemoglobin as compared with the wavelength band for blue in the second filter group F2, in an image obtained by radiating light in the wavelength band for blue in the first filter group F1, the influence of the absorption by blood vessels is low, and the absorption by fat tissue is high. On the other hand, an image that is obtained by separately capturing reflected light beams of the beams that have passed through the individual filters B2, G2, and R2 in the second filter group F2 and by combining the captured beams after imparting the corresponding colors thereto forms a white-light image.

Because the wavelength band for green G1 in the first filter group F1 is a region in which the absorption by hemoglobin occurs instead of the absorption by R-carotene, a region in which the intensity is low in an image obtained by radiating the light in the wavelength band for green G1 in the first filter group F1 indicates a region in which blood exists, for example, blood vessels.

Because both the absorption by R-carotene and that by hemoglobin do not occur in the wavelength band for red R1 in the first filter group F1, an image obtained by radiating the light in the wavelength band for red R1 in the first filter group F1 shows morphological features of the biological tissue surface.

The image-processing portion 17 performs image processing for combining the image signals stored in the memory 55 after imparting different colors thereto.

The control portion 18 synchronizes the timing at which images are captured by the image-acquisition device 51, rotation of the filter turret 52, and the timing at which the image processing is performed by the image-processing portion 17.

In the thus-configured biological observation apparatus 50 according to this embodiment, first, the second filter group F2 in the filter turret 52 is moved into the optical axis of the light coming from the xenon lamp 12, the illumination light beams in blue B2, green G2, and red R2 are sequentially radiated, and the reflected light beams at the observation subject site when the respective illumination light beams are radiated are sequentially captured by the image-acquisition device 51.

The sets of image information corresponding to the illumination light beams of the respective colors are sequentially stored in the memory 55, and the sets of image information are transmitted to the image-processing portion 17 from the memory 55 at the point in time at which the sets of image information corresponding to the three types of the illumination light beams, that is, those in blue B2, green G2, and red R2, are acquired. In the image-processing portion 17, the pre-processing portion 21 and the post-processing portion 22 perform the respective types of image processing, and, the post-processing portion 22 applies the colors of the illumination light beams radiated when the sets of image information are captured to the sets of image information, and combines the sets of image information. By doing so, the white-light image is generated, and the generated white-light image is transmitted to the image-displaying portion 6 via the display-form setting portion 26 and is displayed on the image-displaying portion 6.

In the white-light image, for example, in the regions in which blood vessels exist, because absorptions occur in the wavelength bands in blue B2 and green G2, blood vessels are displayed in red. In the regions in which fat exists, because absorption occurs in blue B2, fat is displayed in yellow. However, in the case in which fat tissue is extremely thin, the color of blood vessels in an organ behind the fat tissue passes therethrough, thus making it difficult to ascertain the presence of the fat tissue.

Therefore, in such a case, the first filter group F1 in the filter turret 52 is moved to the position at which the first filter group F1 is disposed on the optical axis of the light coming from the xenon lamp 12, the illumination light beams in blue B1, green G1, and red R1 are sequentially radiated, and the reflected light beams reflected at the observation subject site when the respective illumination light beams are radiated are sequentially captured by the image-acquisition device 27.

As with when capturing the white-light image, the sets of the image information corresponding to the illumination light beams of the respective colors are sequentially stored in the memory 55, and the three-channel image signals are transmitted to the image-processing portion 17 at the point in time when the sets of image information corresponding to the three types of the illumination light beams, that is, those in blue B1, green G1, and red R1, are acquired.

Image processing performed in the image-processing portion 17 is similar to that in the first embodiment.

As has been described above, with the system with which the three-channel image signals are sequentially acquired by using the monochromatic CCD 51 also, as with the system with which the three-channel image signals are simultaneously acquired by using the color CCD 11, it is also possible, in the special-light observation mode, to display thin fat that exists on surfaces of an organ or other types of tissue such as connective tissue or the like in a conspicuous manner.

The above-described embodiment can be modified as below.

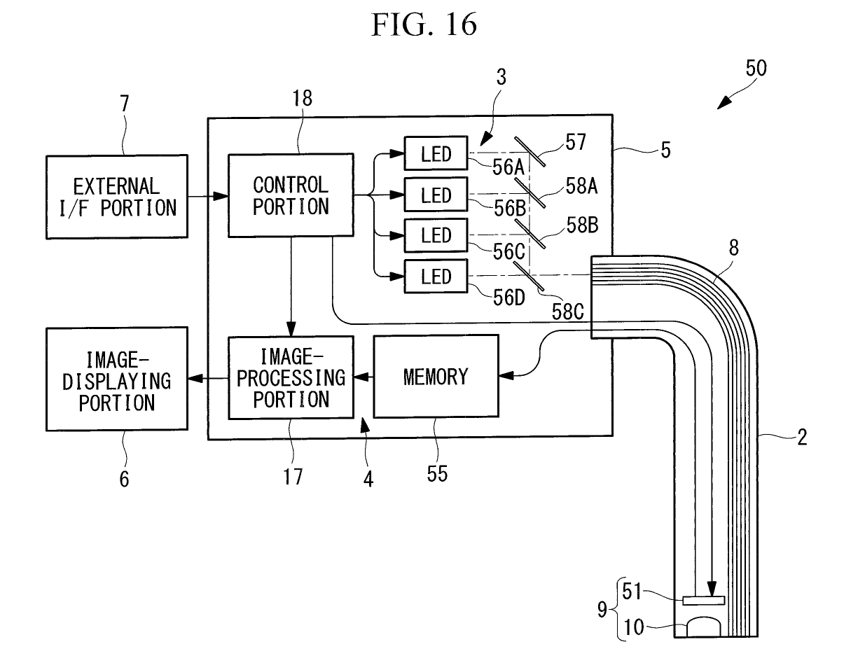

In the above-described embodiment, the light-source portion 3 sequentially emits beams in the different wavelength bands by means of the xenon lamp 12 and the filter turret 13. As shown in FIG. 16, as a first modification, a plurality of light emitting diodes (LEDs) 56A, 56B, 56C, and 56D that emit beams in the different wavelength bands may be disposed so that the beams coming therefrom can be made to enter the same light-guide cable 7 by means of a mirror 57 and dichroic mirrors 58A, 58B, and 58C.

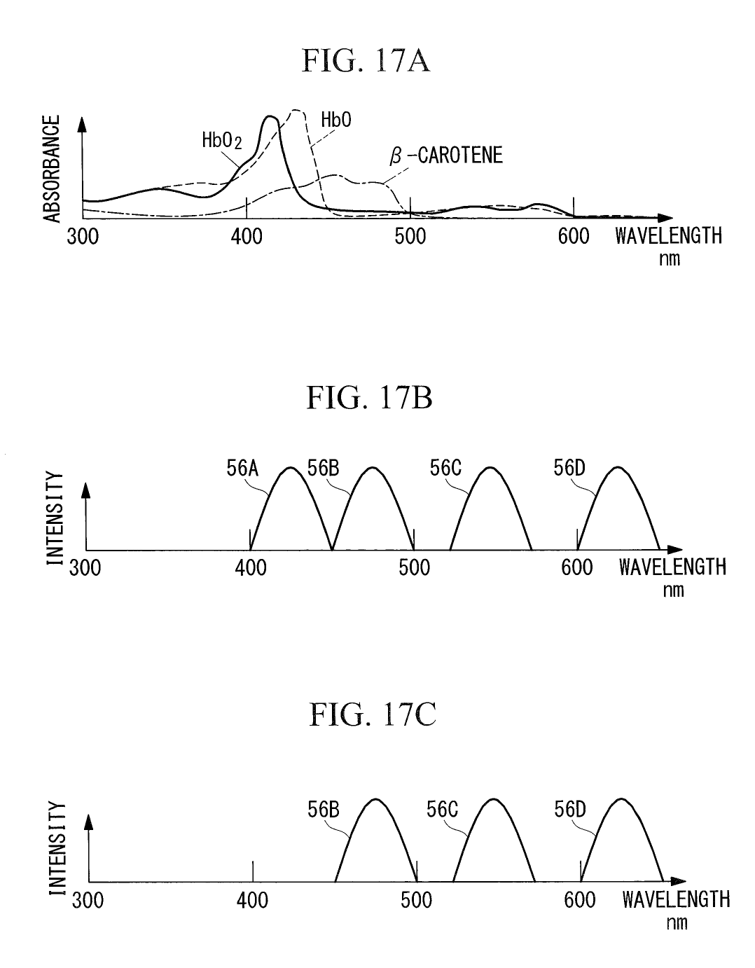

In the example shown in FIG. 16, the four light emitting diodes 56A to 56D in the wavelength bands in ranges of 400 to 450 nm, 450 to 500 nm, 520 to 570 nm, and 600 to 650 nm are provided. In the white-light observation mode, as shown in FIG. 17B, the beams from the light emitting diodes 56A and 56B in the range of 400 to 500 nm may be used as the blue illumination light beams, the beam from the light emitting diode 56C in the range of 520 to 570 nm may be used as the green illumination light beam, and the beam from the light emitting diode 56D in the range of 600 to 650 nm may be used as the red illumination light beam. On the other hand, in the special-light observation mode, as shown in FIG. 17C, the beam from the light emitting diode 56B in the range of 450 to 500 nm may be used as the blue illumination light beam.

As a result, as with the biological observation apparatus 1 in FIG. 1, it is possible, in the special-light observation mode, to display thin fat that exists on surfaces of an organ in a conspicuous manner. FIGS. 17A and 2A show the same graph.

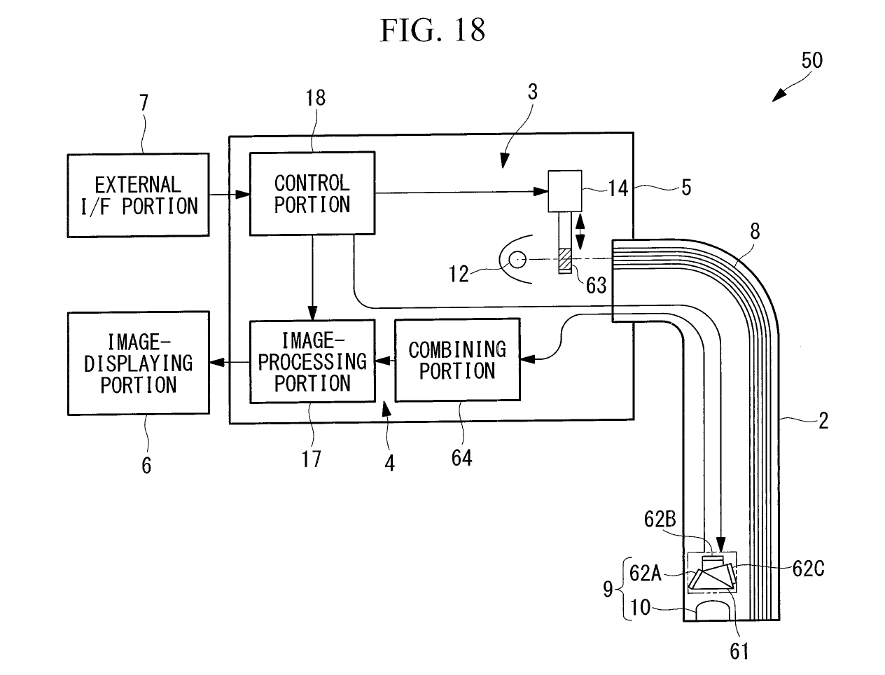

As shown in FIG. 18, as a second modification, it is permissible to employ a 3 CCD system provided with a color separation prism 61 that diffracts the reflected light beams returning from the imaging subject for the separate wavelength bands, and three monochromatic CCDs 62A, 62B, and 62C that capture beams in the respective wavelength bands.

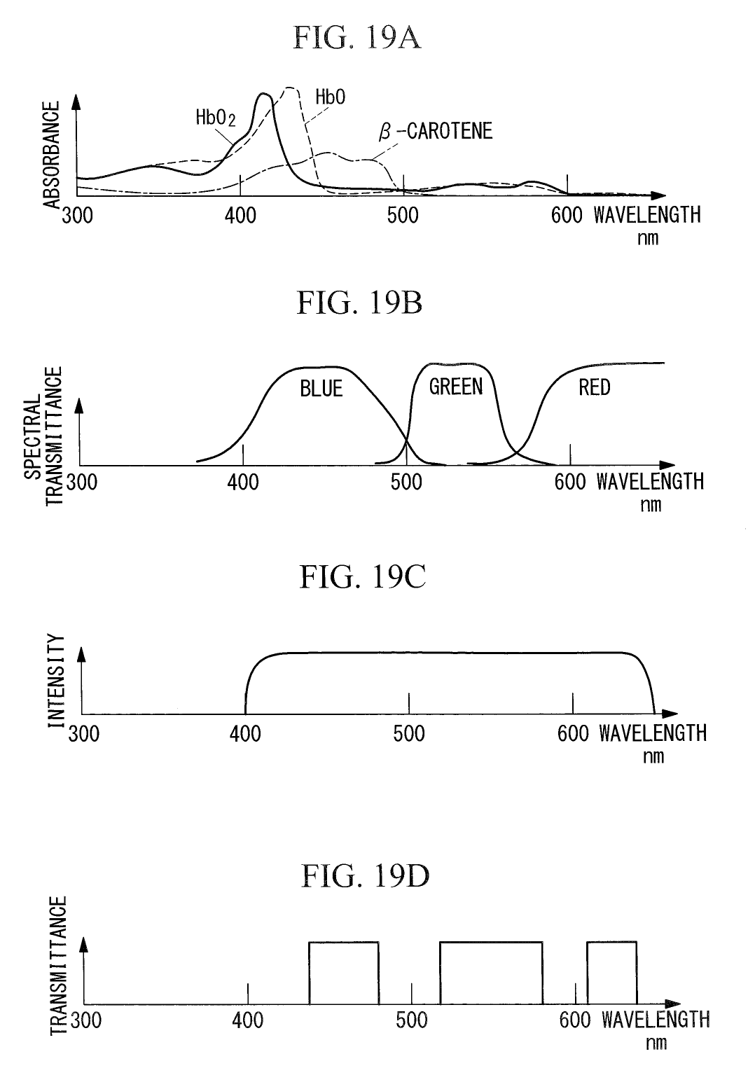

The color separation prism 61 diffracts, for the separate wavelength bands, the reflected light beams coming from the imaging subject according to the transmittance characteristics shown in FIG. 19B. FIGS. 19A and 2A are the same graph. FIGS. 19C and 2C are also the same graph.

In this case also, it is permissible to provide, instead of the filter turret 13, a filter 63 that can be inserted into and retracted from the optical axis of the light coming from the xenon lamp 12 by means of the linear motion mechanism 14. As shown in FIG. 19D, the filter 63 allows beams in three desired wavelength bands to pass therethrough and blocks beams in other wavelength bands.

The filter 63 is retracted from the optical axis in the white-light observation mode, and, the filter 63 is inserted into the optical axis in the special-light observation mode. The images acquired by the respective monochromatic CCDs 62A to 62C are converted to the three-channel form by a combining portion 64 and are output to the image-processing portion 17. By doing so, as with the biological observation apparatus 1 in FIG. 1, it is also possible, in the special-light observation mode, to display thin fat that exists on surfaces of an organ or other types of tissue such as connective tissue or the like in a conspicuous manner.

As a third modification, a magnification switching portion (not shown) that switches the observation magnification may be provided; and the observation mode may be switched to the special-light observation mode when the observation magnification is switched to a high magnification, and the observation mode may be switched to the white-light observation mode when the observation magnification is switched to a low magnification. By employing the special-light observation mode during the high-magnification observation, it is possible to perform precision treatment while checking the boundary between fat and other tissue, and, by employing the white-light observation mode during the low-magnification observation, it is possible to roughly observe the entire site to be treated.

In the above-described embodiment, the confidence-calculating portion 25 calculates the confidence for the fat-region information that indicates the fat regions set by identifying, by means of the threshold processing, the local regions in which the values of the fat image signals are sufficiently high. As a fourth modification, the confidence-calculating portion 25 may calculate the confidence for the fat-region information that indicates the fat regions in the entire screen.

For example, the average, the median, or the maximum of the calculated confidences of the individual sets of the fat-region information may be used as the calculated confidence for the fat-region information that indicates the fat regions in the entire screen. The display-form setting portion 26 may perform manipulating processing for displaying (notifying) an alert on the basis of the calculated confidence for the fat-region information indicating the fat regions in the entire screen. For example, in the case in which confidence for fat detection is low over the entire screen, an alert for notifying the surgeon about the low confidence may be displayed.

In the case in which the calculated confidence for the fat-region information is low, the control portion 18 may switch the light-source setting of the light-source portion 3 to the white light. Because the white light is brighter as compared with the special light, a brighter image is obtained by the image-acquisition device 11 as compared with a case of the illumination light in a specific wavelength band. Therefore, the surgeon performing treatment such as washing off blood or the like thus making it easier to remove hindering factors that cause the fat detection precision to be deteriorated. Because it is difficult to visually ascertain fat when the light source is switched to the white light, fat detection processing may be stopped.

As above, although the individual embodiments of the present invention and the modifications thereof have been described in detail with reference to the drawings, the specific configurations are not limited to these embodiments, and design alterations or the like within a range that does not depart from the scope of the present invention are also encompassed. For example, the biological observation apparatuses 1 and 50 according to the present invention are not limited to endoscopes, and it is possible to widely apply them to apparatuses for observing biological bodies, such as a biological observation apparatus or the like employed in robot surgery.

As a result, the following aspect is read by the above described embodiment of the present invention.

A first aspect of the present invention is an image-processing apparatus including: a fat-region-information detecting portion that detects fat-region information that indicates a fat region in which fat exists in a biological-tissue image; a blood-region-information detecting portion that detects blood-region information that indicates a blood region in which blood exists in the biological-tissue image, a confidence-calculating portion that calculates a confidence for the fat-region information on a basis of the fat-region-information detected by the fat-region-information detecting portion and the blood-region-information detected by the blood-region-information detecting portion; and a display-form manipulating portion that manipulates the fat region indicated by the fat-region information for which the calculated confidence calculated by the confidence-calculating portion is lower than a reference confidence that serves as a reference so as to have a display form that can be distinguished from a peripheral region.

With the first aspect of the present invention, the fat-region-information detecting portion detects the fat-region information in the input biological-tissue image, and the confidence-calculating portion calculates the confidence (calculated confidence) of that fat-region information. During surgery, there are cases in which fat-region information cannot be accurately detected because fat detection is hindered due by the influence of blood existing on an imaging subject and disturbances such as an insufficient exposure level, bright spots, mist, forceps, or the like.

In response, by manipulating, by means of the display-form manipulating portion, the fat region indicated by the fat-region information for which the calculated confidence is lower than the reference confidence that serves as a reference in the biological-tissue image so as to have the display form that can be distinguished from the peripheral region, it is possible to allow a surgeon to ascertain the fat region indicated by the fat-region information that cannot be accurately detected because fat detection is hindered due by the influence of blood and disturbances. By doing so, the surgeon can perform treatment such as removing blood and disturbances in that fat region, and thus, it is possible to reduce the risk of damaging the nerves.

In the above-described aspect, the calculated confidence may be increased with an increase in an SN ratio of the fat-region information and may be decreased with a decrease in the SN ratio.

The SN ratio of the fat-region information is decreased with an increase in the influence of blood and disturbances, and the SN ratio of the fat-region information is increased with a decrease in the influence of blood and disturbances. Therefore, by employing the above-described configuration, it is possible to calculate an accurate confidence for the fat-region information on the basis of the SN ratio of the fat-region information by means of the confidence-calculating portion.

In the above-described aspect, the calculated confidence may be increased with a decrease in a proportion of the blood-region information with respect to the fat-region information, and may be decreased with an increase in the proportion.

Because a large amount of blood tends to exist on the imaging subject during surgery due to bleeding or the like, the detection of the fat-region information is often hindered by blood. An increasing amount of blood exists in the fat region with an increase in the proportion of the blood-region information relative to the fat-region information, and thus, the detection of the fat-region information is hindered; and a decreasing amount of blood exists in the fat region with a decrease in the proportion of the blood-region information relative to the fat-region information, and thus, the detection of the fat-region information is not hindered. Therefore, by employing the above-described configuration, it is possible to obtain a more accurate confidence for the fat-region information on the basis of the proportion of the blood-region information relative to the fat-region information by means of the confidence-calculating portion.

In the above-described aspect, the display-form manipulating portion may display the fat region indicated by the fat-region information for which the calculated confidence is lower than the reference confidence in an emphasized manner as compared with the peripheral region. The display-form manipulating portion may display the peripheral region in an emphasized manner as compared with the fat region indicated by the fat-region information for which the calculated confidence is lower than the reference confidence.

By employing such a configuration, in both cases, it is possible to clearly distinguish the fat region indicated by the fat-region information for which the confidence is low from the peripheral region by using simple methods.

In the above-described aspect, the display-form manipulating portion may notify a surgeon about the fat region indicated by the fat-region information for which the calculated confidence is lower than the reference confidence.

By employing such a configuration, the surgeon can more easily ascertain the presence of the fat region indicated by the fat-region information for which the confidence is low.

A second aspect of the present invention is a biological observation apparatus including: an irradiating portion that can radiate illumination light onto biological tissue; an image-acquisition portion that captures, of reflected light, which is the illumination light radiated from the irradiating portion and reflected at the biological tissue, reflected light in a specific wavelength band, thus acquiring the biological-tissue image; any of the above-described image-processing apparatuses that process the biological-tissue image acquired by the image-acquisition portion; and a display portion that displays the biological-tissue image processed by the image-processing apparatus.

With the second aspect of the present invention, the irradiating portion radiates the illumination light onto the biological tissue, and, of the reflected light reflected at the biological tissue, the image-acquisition portion captures the reflected light in a specific wavelength band. For example, of the reflected light reflected at the biological tissue, by capturing, by means of the image-acquisition portion, the reflected light in a specific wavelength band in which the influences due to the presence of blood vessels are low and the influences due to the presence of fat is high, it is possible to acquire a biological-tissue image that is subjected to an influence due to the presence of fat.

With respect to the thus-acquired biological-tissue image, the image-processing apparatus detects the fat-region information, and the fat region indicated by the fat-region information for which the confidence is low is subjected to processing for manipulating the fat region so as to have a display form that can be distinguished from the peripheral region, thus being displayed on the display portion. Therefore, even in the case in which accurate detection cannot be performed because fat detection is hindered due by the influence of blood and disturbances, it is possible to reduce the risk of damaging the nerves by means of treatment performed by the surgeon by allowing the surgeon to ascertain the fat region indicated by the fat-region information for which the confidence is low.

The above-described aspect may be provided with a control portion that causes white light to be emitted as illumination light to be radiated onto the biological tissue by the irradiating portion in the case in which the calculated confidence is lower than the reference confidence and that causes the image-acquisition portion to capture reflected light of the white light itself reflected at the biological tissue.

By employing such a configuration, because the white light is brighter as compared with illumination light in a specific wavelength band, a brighter image is obtained by the image-acquisition portion as compared with the case of the illumination light in the specific wavelength band. Therefore, the surgeon performing treatment such as washing off blood or the like thus making it easier to remove hindering factors that cause the fat detection precision to be deteriorated. Because it is difficult to visually ascertain fat when the light source is switched to the white light, fat detection processing may be stopped.

A third aspect of the present invention is an image-processing method including: a fat-region-information detecting step of detecting fat-region information that indicates a fat region in which fat exists in a biological-tissue image; a blood-region-information detecting step of detecting blood-region information that indicates a blood region in which blood exists in the biological-tissue image; a confidence calculating step of calculating a confidence for the fat-region information on a basis of the fat-region-information detected in the fat-region-information detecting step and the blood-region-information detected by the blood-region-information detecting step; and a display-form manipulating step of manipulating the fat region indicated by the fat-region information for which the calculated confidence calculated in the confidence calculating step is lower than the reference confidence so as to have a display form that can be distinguished from a peripheral region.

With the third aspect of the present invention, in the case in which blood exists on the imaging subject or in the case in which there are influences of disturbances such as an insufficient exposure level, bright spots, mist, forceps, or the like, it is possible to easily process the biological-tissue image so that the surgeon can distinguish, from the peripheral region, the fat region that cannot be accurately detected because fat detection is hindered.

### REFERENCE SIGNS LIST

1, 50 biological observation apparatus
3 light-source portion (irradiating portion)
6 image-displaying portion (display portion)
9 imaging optical system (image-acquisition portion)
17 image-processing apparatus (image-processing portion)
18 control portion
26 display-form setting portion (display-form manipulating portion)
32 fat-region setting portion (fat-region-information detecting portion)
34 blood-region setting portion (blood-region-information detecting portion)
37 fat-region confidence-calculating portion (confidence-calculating portion)
SB3 fat-region-information detecting step
SB7 confidence calculating step
SB9 display-form manipulating step

1. An image-processing apparatus comprising:
a fat-region-information detecting portion that detects fat-region information that indicates a fat region in which fat exists in a biological-tissue image;
a blood-region-information detecting portion that detects blood-region information that indicates a blood region in which blood exists in the biological-tissue image;
a confidence-calculating portion that calculates a confidence for the fat-region information on a basis of the fat-region-information detected by the fat-region-information detecting portion and the blood-region-information detected by the blood-region-information detecting portion; and
a display-form manipulating portion that manipulates the fat region indicated by the fat-region information for which the calculated confidence calculated by the confidence-calculating portion is lower than a reference confidence that serves as a reference so as to have a display form that can be distinguished from a peripheral region.

2. An image-processing apparatus according to claim 1, wherein the calculated confidence is increased with an increase in an SN ratio of the fat-region information and is decreased with a decrease in the SN ratio.

3. An image-processing apparatus according to claim 1,
wherein the calculated confidence is increased with a decrease in a proportion of the blood-region information with respect to the fat-region information, and is decreased with an increase in the proportion.

4. An image-processing apparatus according to claim 1, wherein the display-form manipulating portion displays the fat region indicated by the fat-region information for which the calculated confidence is lower than the reference confidence in an emphasized manner as compared with the peripheral region.

5. An image-processing apparatus according to claim 1, wherein the display-form manipulating portion displays the peripheral region in an emphasized manner as compared with the fat region indicated by the fat-region information for which the calculated confidence is lower than the reference confidence.

6. An image-processing apparatus according to claim 1, wherein the display-form manipulating portion notifies a surgeon about the fat region indicated by the fat-region information for which the calculated confidence is lower than the reference confidence.

7. A biological observation apparatus comprising:
an irradiating portion that can radiate illumination light onto biological tissue;
an image-acquisition portion that captures, of reflected light, which is the illumination light radiated from the irradiating portion and reflected at the biological tissue, reflected light in a specific wavelength band, thus acquiring the biological-tissue image;
an image-processing apparatus according to claim 1 that processes the biological-tissue image acquired by the image-acquisition portion; and
a display portion that displays the biological-tissue image processed by the image-processing apparatus.

8. A biological observation apparatus according to claim 7, further comprising:
a control portion that causes white light to be emitted as illumination light to be radiated onto the biological tissue by the irradiating portion in the case in which the calculated confidence is lower than the reference confidence and that causes the image-acquisition portion to capture reflected light of the white light itself reflected at the biological tissue.

9. An image-processing method comprising:
a fat-region-information detecting step of detecting fat-region information that indicates a fat region in which fat exists in a biological-tissue image;
a blood-region-information detecting step of detecting blood-region information that indicates a blood region in which blood exists in the biological-tissue image;
a confidence calculating step of calculating a confidence for the fat-region information on a basis of the fat-region-information detected in the fat-region-information detecting step and the blood-region-information detected by the blood-region-information detecting step; and
a display-form manipulating step of manipulating the fat region indicated by the fat-region information for which the calculated confidence calculated in the confidence calculating step is lower than the reference confidence so as to have a display form that can be distinguished from a peripheral region.
